(19) United States
(12) Patent Application Publication
(10) Pub. No.: US 2021/0059849 A1
(43) Pub. Date: Mar. 4, 2021

(54) CUSTOMIZABLE WEARABLE TOP TO TREAT SCOLIOSIS IN A PERSON

(71) Applicant: THE HONG KONG POLYTECHNIC UNIVERSITY, Hong Kong (CN)

(72) Inventors: Yiu-Wan Joanne YIP, Hong Kong (CN); Ho-Yi Olivia FUNG, Hong Kong (CN); Kit-Lun YICK, Hong Kong (CN); Chi-Yung TSE, Hong Kong (CN); Man-Chee Kenneth CHEUNG, Hong Kong (CN); Yat-Hong Kenny KWAN, Hong Kong (CN); Pui-Yin Jason CHEUNG, Hong Kong (CN); Sun-Pui NG, Hong Kong (CN)

(21) Appl. No.: 16/551,832

(22) Filed: Aug. 27, 2019

(51) Int. Cl. A61F 5/02

(52) U.S. Cl. CPC A61F 5/026

(57) ABSTRACT

The invention relates to a customizable wearable top for the treatment of scoliosis, such as Adolescent Idiopathic Scoliosis (AIS), in a person and related methods. In one form there is provided a customizable wearable top to treat scoliosis in a person comprising: a body portion formed from a soft material and being wearable around the upper torso and having a body front and a body back; a strap adjuster on an outward facing surface of the body back, the strap adjuster configured to, or customizable to, mount a first end of one or more straps at a selectable longitudinal position thereon, such that when worn the straps are extendable around at least a portion of a wearer's upper torso to apply a bias force to a treatment site on the person corresponding to the selectable longitudinal position.

### FIELD

The invention relates to a customizable wearable top for the treatment of scoliosis, such as Adolescent Idiopathic Scoliosis (AIS), in a person.

### BACKGROUND

Scoliosis is generally defined as the three-dimensional deformity of spine, thorax and trunk. A diagnosis is made when the spine deviates laterally with a Cobb angle of 10° or more. The majority of scoliosis cases are idiopathic, in which no known cause can be found with a multifactorial etiology. It occurs mainly in adolescent, namely Adolescent Idiopathic Scoliosis (AIS), during the period of growth spurt from the age of 10 until the reach of skeletal maturity. To halt the progression of AIS and avoid surgery, bracing is the most common non-operative treatment option for adolescents with a spinal curve of 25° to 40°. The external mechanical forces exerted by braces restore the alignment of spine and body contour. However, there are two main problems associated with the existing braces which affect the treatment outcomes: one is the long production lead time for scoliosis brace and the second is the low brace compliance in patients.

Scoliosis braces are custom-made in accordance with the spinal curvature and body shape of each patient. Treating orthotist is responsible throughout the fabrication process from shape capturing, design, fitting to follow-up adjustments, as trained skills are required to ensure a well-designed orthosis that fits well on patient. Techniques and ways to fabricate braces have been evolving from plaster-cast to prefabrication and to CAD/CAM modelling; however, the required production lead time is still long. Existing fabrication methods include plaster casting, prefabrication, computer-aided-design/computer-aided-manufacturing (CAD/CAM), and simulation of brace fitting. Each of these is briefly discussed below.

Plaster casting is a traditional method introduced by French surgeon Yves Cotrel and American physician Joseph Risser. With the use of a casting frame, patient is put in a supine position with traction and corrective force applied. Plaster or synthetic bandage is then wrapped around the torso until it hardens, and a corrected trunk impression of patient is captured upon bandage removal. By hand-modifying the obtained plaster mold, the brace is customized and fabricated according to the brace type.

The concept of prefabrication later emerged when Dr. John Hall and an orthotist, William Miller, noticed that many plaster models shared a similar shape and size. Prefabricated symmetric brace modules were thus created in several different sizes, and patients were fitted in accordance with their body measurements in terms of circumference, width and depth. Customized pads and trim lines were applied, in correspondence with the spinal situation of patients, to provide corrective forces based on the bracing principles. Under the prefabrication concept, orthosis is fabricated and customized over a standard body form rather than a specific patient.

Computer-aided Design/Computer-aided Manufacturing (CAD/CAM) can also be used to prepare a brace based on the body shape of patient faster than with casting. The body shape is captured in 3D form with a scanner before uploading to the CAD/CAM software for model preparation. The model can be asymmetrically shifted or balanced based on the mechanical principles of the brace type. Pads or pushes can also be built into the CAD/CAM model, similar to what would have done with the hand-modified models mentioned previously. Before sending off to a milling machine to have the positive model created, the shape of the finished brace can be reviewed by drawing trim lines on the model. Once the positive foam model is ready, plastic is then vacuum-formed, trimmed and smoothed for the initial brace fitting. Fabrication techniques with CAD/CAM are similar to those used for casting, but an electronic record on the body shape of patient can be maintained.

Software has recently been developed for simulating a brace fitting and its corresponding effectiveness prior to fabrication. By importing the 3D body image and radiographs of the patients to the software, a virtual body is automatically created. Orthotist can choose to modify the model or allow the software to generate the most effective brace design for spinal curve reduction. A simulator is built-in for testing the result of the virtual brace on the virtual body. Design can therefore be modified numerous times to ensure an optimum result before fitting onto a real patient.

Although the brace fabrication methods have been enhanced to simplify the process of making, the scoliosis brace is still one-off and custom made for each patient. The production lead time might have shortened; however, patients still have to wait for a few weeks before the brace is ready for their use. To ensure that patients receive brace treatment promptly after the diagnosis of AIS, there is a need to reduce the lead time in production.

Solely the occurrence of AIS has already caused social problems for most patients. In concurrent with brace treatment which further affects the self- and body image, brace compliance becomes low. One of the major concerns why patients refuse to wear a brace is related to appearance. The reason is that patients are in their adolescent years, a period in which body image and peer conformity have high importance. The social stigma conveyed by an unappealing looking brace might set them away from their peers and causes self-devaluation. Other bracing problems that affect the compliance of patients are skin irritation and physical discomfort.

Scoliosis braces are usually divided into two categories as hard braces and soft braces. They all share the same goals, which are to restore the alignment of spine and body contour, to halt the progression of spine and to avoid surgery. The differences mainly lie in the type of material used, fabrication method and biomechanical principles of spinal correction, which respectively have their own strengths and weaknesses. Hard braces are considered effective for preventing spinal curve progression during pubertal growth, but they are rigid, bulky, uncomfortable and aesthetically unappealing to wear. As a result, patient compliance is relatively low, and the negative impacts of hard braces on the quality of life and psychological being of patients are concerned. Soft braces, on the other hand, have been developed to address the shortcomings of hard braces and remedy the problems related to comfort and appearance, but their efficacy in spinal correction remains controversial.

In view of the above, there is a need to improve patient compliance with a better scoliosis brace is perceived. Ideally, the design should maintain low visibility, provide better comfort that existing brace designs, and minimally restrict a patient's movement during daily life.

It is an object of the invention to address one or more of the shortcomings of the prior art.

### SUMMARY OF INVENTION

In a first aspect of the invention, there is provided a customizable wearable top to treat scoliosis in a person comprising: a body portion formed from a soft material and being wearable around the upper torso and having a body front and a body back; a strap adjuster on an outward facing surface of the body back, the strap adjuster configured to, or customisable to, mount a first end of one or more straps at a selectable longitudinal position thereon such that the straps are extendable around at least a portion of a wearer's upper torso to apply a bias force to a treatment site on the person corresponding to the selectable longitudinal position.

By “soft material” it is generally meant that the material is non-rigid and is pliable or otherwise change shape under the force of their own weight. Soft materials include, but are not limited to, natural fibres such as wool or animal hairs, animal skins such as leather, silk, cotton, flax, hemp, and jute etc; and artificial or synthetic fibres formed from materials such as nylons, polyesters, modacrylics, olefins, acrylics, rayon, polyvinylchloride fibres, elastane (polyether-polyurea copolymer), polyvinyl alcohol, aramids, polybenzimidazole, polyphenylene sulfide, lyocell, polylactic acid, etc.

In an embodiment, the strap adjuster has an elongate body that is vertically aligned with the wearable top. Preferably, the elongate body is a panel, wherein the panel comprises or is customisable to comprise a plurality of attachment points to mount the one or more straps at the selectable longitudinal position.

In one form of the above embodiment, the elongate body is located in a central portion of the body back and extends from a body back upper portion to a body back lower portion, such that when worn, the elongate body is aligned over the persons vertebral line and/or extends over at least a length of the wearer's thoracic and lumbar vertebrae.

In an embodiment, the strap adjuster is configured to, or is customizable to, mount the one or more straps threadably therethrough.

In an embodiment, the strap adjuster comprises a plurality of selectable components which either alone or in combination form one or more mount portions or are customizable in combination to form one or more mount portions for attachment of the one or more straps.

In one form of the above embodiment, each selectable component is a hole or a window, and the plurality of selectable components is an array of holes comprising at least two vertically aligned columns of holes.

Preferably, the strap adjuster is cuttable to form a first slit from two or more adjacent holes (or windows) in a first column of the at least two vertically aligned columns, and a second slit from two or more adjacent holes (or windows) in a second column of the at least two vertically aligned columns, and wherein the strap is mountable to the strap adjuster by threading the strap through the first slit and the second slit. Preferably, the strap folds in on itself and fastens to itself about the first slit and the second slit, for example, by using a hook-and-loop fastening arrangement. It is also preferred that the strap adjuster comprises a cuttable portion between adjacent holes that is cuttable to form the first and second slits. The cuttable portion is preferably a thin border between adjacent holes (or windows), and is preferably frangible, formed from thinner material than the bulk of the strap adjuster and/or is otherwise weakened.

In one or more forms, the strap adjuster is formed from a thermoplastic material having a melting point in the range of 130° C. to 180° C. Preferably, the melting point is from 135° C. Most preferably, the melting point is from 140° C. Additionally or alternatively, it is preferred that the melting point is up to 175° C. Most preferably, the melting point is up to 170° C.

In an embodiment the strap adjuster is heat welded to the body back.

In an embodiment, the wearable top further comprises a front attachment structure on an outward facing surface of the body front for attachment of a second end of each of the one or more straps, such that each strap is extendable from the strap adjuster and the front attachment structure.

In an embodiment, the wearable top further comprises the one or more straps. Preferably the wearable top comprises two straps or three straps.

In one form of the above embodiment, each of the one or more straps comprise a reel-type closure system having a turnable dial configured to tighten and/or loosen the straps. Preferably, the reel-type closure is on a second end of each of the one or more straps, such that the reel-type closure is accessible from a body front of the wearable top when worn.

In an embodiment, the body back has a receiving surface for the customized placement of one or more treatment pads and/or mounting structures for retaining one or more treatment pads at a customized location corresponding to the treatment site on the person, e.g. such that when the wearable top is worn the treatment pads are able to apply a corrective force to the treatment site.

In one form of the above embodiment, the receiving surface is for the customized placement of mounting structures for retaining the one or more treatment pads, each mounting structure being a pocket for receiving a treatment, and wherein the receiving surface is formed from a material to which the one or more pockets are heat weldable.

In an embodiment, the wearable top further comprises a plurality of vertically oriented elongate pockets distributed circumferentially around the body portion, each pocket configured to receive a rigid support strut. Preferably, each pocket comprises the rigid support strut therein.

In an embodiment, the strap adjuster further comprises mounting structure for receiving a pelvic belt. Preferably, the wearable top further comprises the pelvic belt.

In one form of the above embodiment, the pelvic belt comprises a receiving surface for mounting one or more treatment pads. Preferably, the one or more treatment pads are mountable to the pelvic belt via a hook-and-loop fastening system.

In one form of the above embodiment, the pelvic belt comprises thigh straps configured to be attached around a wearer's thighs.

In a second aspect of the invention, there is provided the use of the customizable wearable top according to the first aspect of the invention and/or embodiments and/or forms thereof in the treatment of a patient with scoliosis.

In a third aspect of the invention there is provided a method of preparing a customizable wearable top to treat scoliosis in a person, the method comprising: providing a customizable wearable top according to the first aspect of the invention and/or embodiments and/or forms; selecting a selectable longitudinal position based on a scoliosis diagnosis of the person and mounting the first end of the strap to the strap adjuster at the selected longitudinal location.

In an embodiment of the third aspect, the strap adjuster is customizable to mount the first end of one or more straps at the selected longitudinal position, and the method further comprises: customising the strap adjuster to include one or more mounts to mount the first end of the one or more straps at the selected longitudinal position.

In one form of the above embodiment, the strap adjuster is in the form of a vertically oriented panel having an array of holes or windows arranged in at least two columns, and the step of customising the strap adjuster further comprises: cutting a portion of the strap adjuster between two or more adjacent holes in a first column of the at least two vertically aligned columns to form a first slit; cutting a portion of the strap adjuster between two or more adjacent holes in a second column to form a second slit; and threading the strap through the first slit and the second slit to mount the strap to the strap adjuster.

In one embodiment of the third aspect, the method further comprises: attaching one or more treatment pads and/or mounting structures for retaining the one or more treatment pads at a customized location on a body back portion of the wearable top corresponding to the treatment site.

In one form of the above embodiment, the step of attaching the one or more treatment pads and/or mounting structures comprises heat welding the one or more treatment pads and/or mounting structures to the body back portion.

### BRIEF DESCRIPTION OF DRAWINGS

FIG. 1a is a photograph of a front view of the customizable wearable top according to one embodiment of the invention.

FIG. 1b is a photograph of a rear view of the customizable wearable top according to one embodiment of the invention.

FIG. 2a is a SolidWorks illustration of the strap adjusted according to one embodiment of the invention.

FIG. 2b is a photograph of the strap adjuster with two slits trimmed into the strap adjuster to accommodate a strap.

FIG. 2c is a photograph showing a strap threadably mounted to the strap adjusted via the two slits.

FIG. 3 is a photograph showing a rigid support strut inserted into an elongate pocket of the customizable wearable top.

FIG. 4a is a photograph showing a pelvic belt attached to the customizable wearable top via attachment to a lower portion of the strap adjuster.

FIG. 4b is an image of the HICKIES® laces.

FIG. 4c is a photograph showing a person wearing the pelvic belt, and in particular, illustrating the leg straps for tightening the pelvic belt around a person's thigh.

FIG. 4d is a photograph of a front view of the pelvic belt.

FIG. 5 is a photograph of a pad holder.

FIG. 6 is a photograph showing the inside surface of the customizable wearable top, and in particular, illustrating the pad holders retaining the treatment pads.

FIG. 7a is an illustration of the bodice component of the customizable wearable top.

FIG. 7b is a close up of the bodice showing in FIG. 7a illustrating the knitted net structure.

FIG. 8a shows the front side (top), rear side (middle) and inner construction (bottom) of the Boa® closure system

FIG. 8b is a photograph of the V-tape hook fastener.

FIG. 8c is a photograph showing the second strap end with Velcro loop tapes sewn on both sides and clipped in between the v-tape hook fastener.

FIG. 8d is a photograph of the second strap end showing the Velcro loop tape printed with different gradations.

FIG. 9 is an illustration of the three-point system principle (left); majority SOSORT consent on pad placed at the level of apical vertebra (middle); majority SOSORT consent on an oblique force vector from dorso-lateral to ventro-medial produced by pad (right).

FIG. 10 illustrates the position of T1 vertebra and transitional point (TP) on central sacral line (CSL); counter-tilting relationship between L4 vertebra and L5 vertebra in the frontal plane

### DESCRIPTION OF EMBODIMENTS

The invention relates to a customizable wearable top for the treatment of scoliosis, such as Adolescent Idiopathic Scoliosis (AIS), in a person and related methods.

In particular, the customizable wearable top includes a body portion or bodice formed from a soft material with a strap adjuster affixed (such as via a heat weldable material) or otherwise attached to a body back portion thereof. The strap adjuster allows the attachment of one or more straps to the body portion at a custom chosen longitudinal position on the body portion such that when worn, the strap extends around a portion of a wearer's upper torso to apply a bias force to a treatment site on the person corresponding to that chosen longitudinal position.

In preferred forms, the customizable wearable top further comprises one or more treatment pads heat welded to an inner surface of the body portion at a custom chosen position corresponding to the treatment site, such that when the wearable top is worn, the straps overlie the treatment pad and apply the bias force thereto such that the treatment pads apply a corrective force to the wearer's scoliotic spine.

The adoption of a strap adjuster for the customized, quick, and easy attachment of straps provides the wearable top with a number of advantages over the hard and soft braces of the prior art (discussed in the background section).

One advantage is that such structure provides a top that takes the best elements of both hard and soft braces whilst avoiding significant problems of each, for example since the body portion is formed from a soft material (e.g. a knitted or woven material) it is more comfortable to wear and is more visually appealing than hard braces thus improving patient compliance.

Likewise, since the top is customizable to include elements of hard braces (such as treatment pads) the top can treat scoliotic spines more effectively than soft braces which have questionable treatment efficacy for treating scoliotic spines with a curvature of >20°. In contrast, the wearable top of the invention is suitable to treat a scoliotic spine with curvature of >20°.

Another advantage is the speed at which the wearable top can be prepared for a patient. The strap adjuster can be easily and quickly modified to mount the straps at the appropriate longitudinal position. Likewise, the pockets for retaining the treatment pads can be quickly positioned within the body portion through heat welding. This contrast particularly with hard braces which require the costly and time-consuming preparation from molds or computer modelling.

The invention is described below in relation to one embodiment thereof.

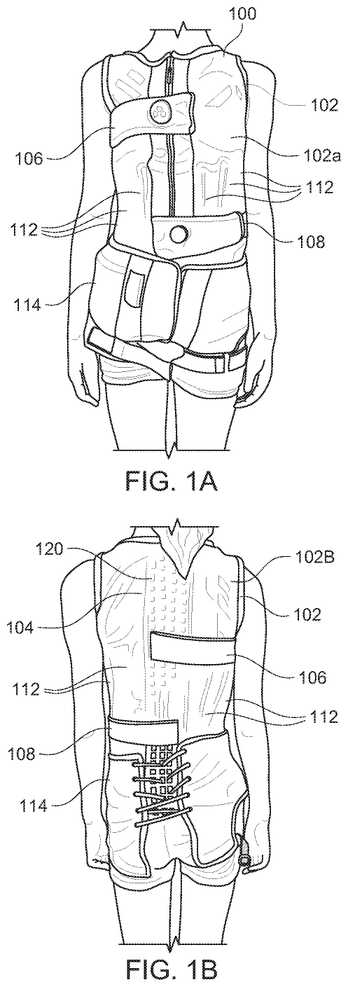

FIG. 1(a) and FIG. 1(b) are illustrations showing front view and rear views of a customizable wearable top 100 for the treatment of Adolescent Idiopathic Scoliosis (AIS) according to one embodiment of the present invention.

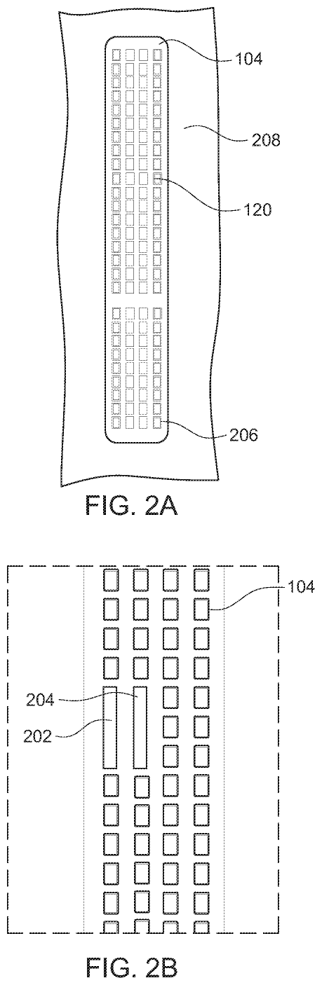

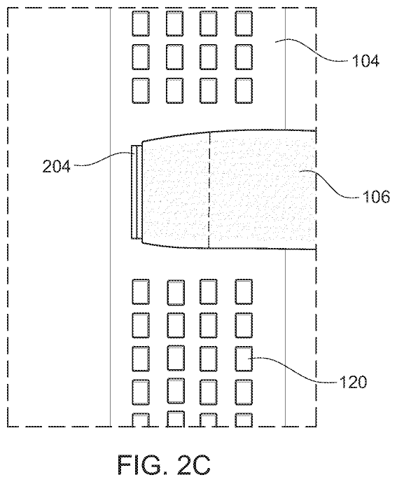

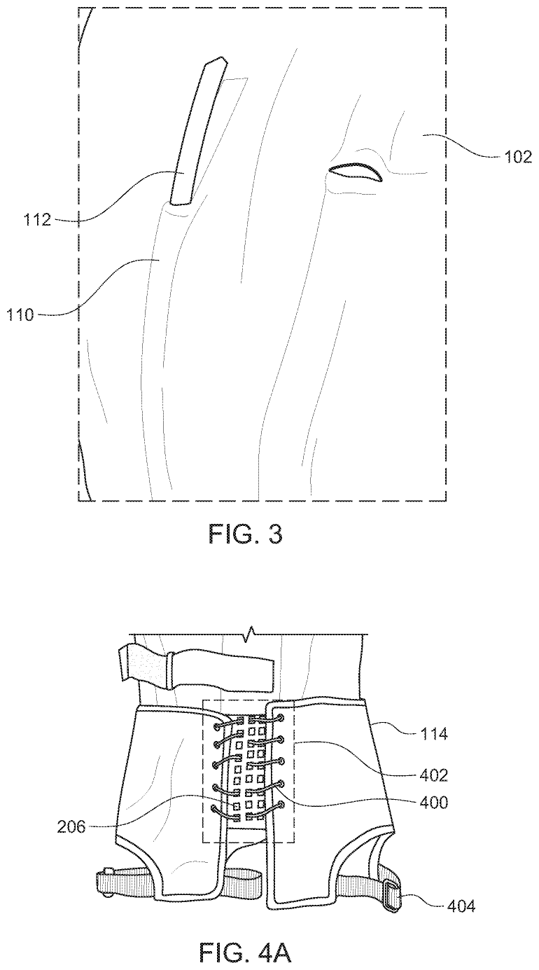

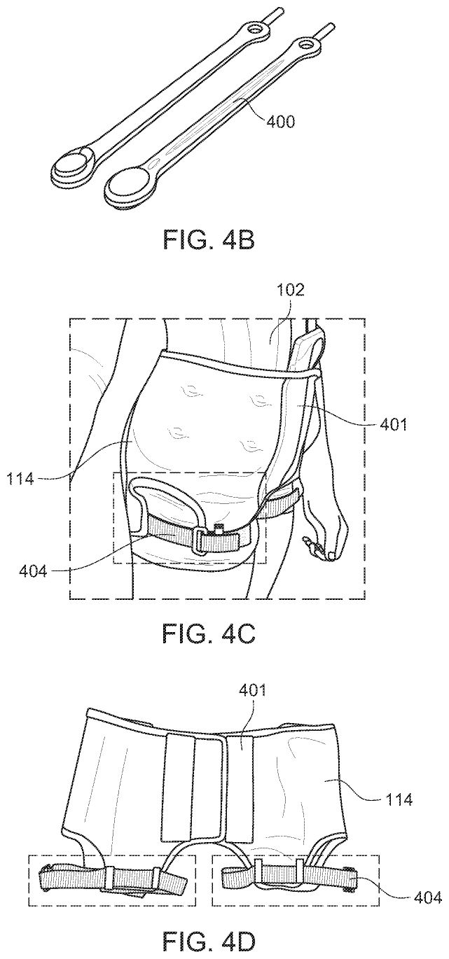

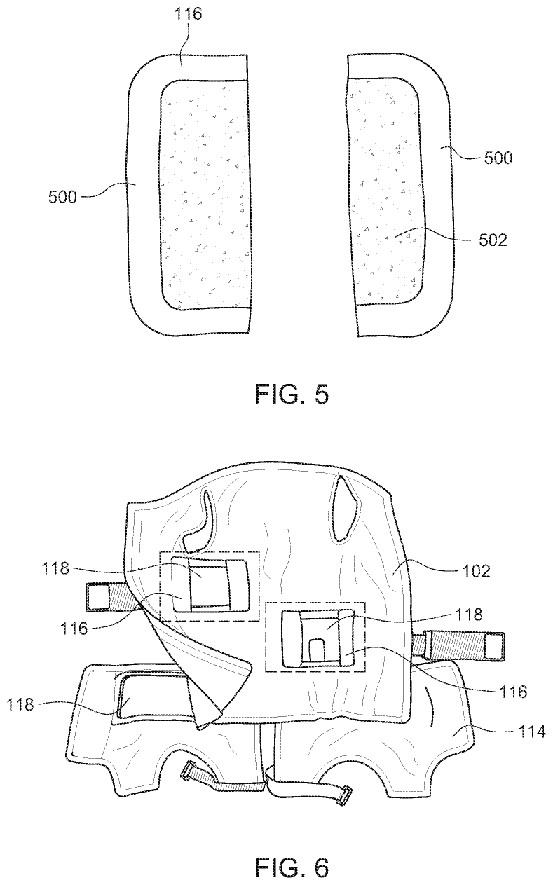

In this embodiment, wearable top 100 is in the form of a soft tight-fitting knit bodice 102 which has a number of components fitted thereto, including a strap adjuster 104 (best shown in FIGS. 2a-2c) for receiving straps 106 and 108, elongate pockets 110 for receiving rigid support struts 112 (as best shown in FIG. 3), pelvic belt 114 (as best shown in FIGS. 4a-4d), and pad holders 116 (see FIG. 5) for retaining treatment pads 118 on an inside surface of the bodice 102 (as best shown in FIG. 6). The components of the top 100 can be pre-fabricated in mass so that customization of the top 100 based on the spinal curvature and body type of a patient can be realised within a short time period. Each of the components of the top 100 is discussed in more detail below.

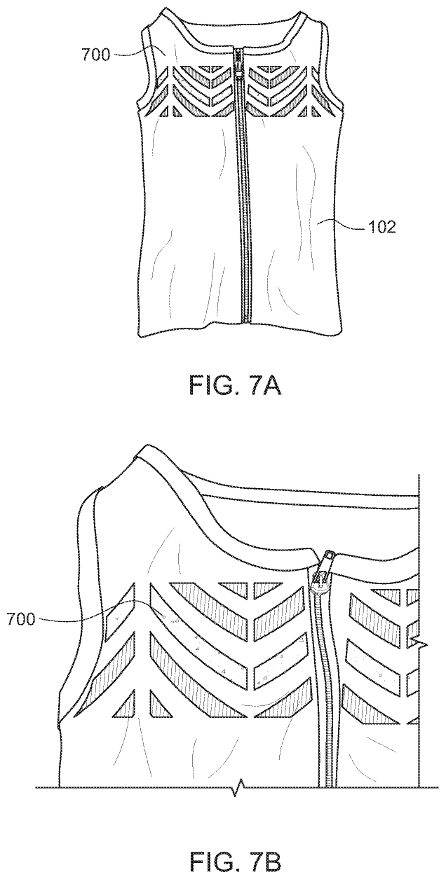

In the illustrated embodiment, the knitted structure of the bodice 102 is double jersey with an additional insertion of elastic inlaid yarns in between. The body front 102a and the body back 102b of the double jersey are respectively knitted using deodorization yarn and cool-to-touch yarn. The cool-to-touch yarn knitted on the body back 102b of the bodice 102 is in contact with the wearer's skin, and thus provides a cool sensation to the wearer. The deodorization yarn provides deodorization, anti-microbial, and moisture management functions, which help the wearable top 100 to stay dry and fresh, especially during summer. To further enhance the comfort of wearer, part of the bodice 102 is knitted in a net structure 700, as shown in FIG. 7a with FIG. 7b showing a close-up view of the knitted net structure, to achieve better breathability. As can be seen, the bodice 102 is similar in appearance to an ordinary top. This helps to reduce the appearance profile of the wearable top 100 which is beneficial since it reduces the stigma and negative emotional impact of a person wearing the top 100 thus improving compliance.

The wearable top 100 has a strap attachment structure 104 adhered to an outward facing surface on the body back 102b of the bodice 102 to which a first end 106a, 108a of straps 106 and 108 are mounted at a customized longitudinal or vertical height on the top 100. This customization is important so that the wearable top 100 can be quickly and readily prepared for a patient and be customized to fit their body size and treat the nature of their spinal curvature. Once the first ends of straps 106 and 108 have been mounted to the strap adjuster 104, straps 106 and 108 are at least partially extended around the wearer's torso and the second end 106b, 108b of straps 106 and 108 are connected to a body front portion 102a of the bodice 102. Straps 106 and 108 are tightenable around the upper torso of the person (and over treatment pads 118 when incorporated into the top 100) such that straps 106 and 108 apply a bias force to the treatment pads 118 which in turn apply a corrective force at treatment locations on the wearer to treat the scoliosis.

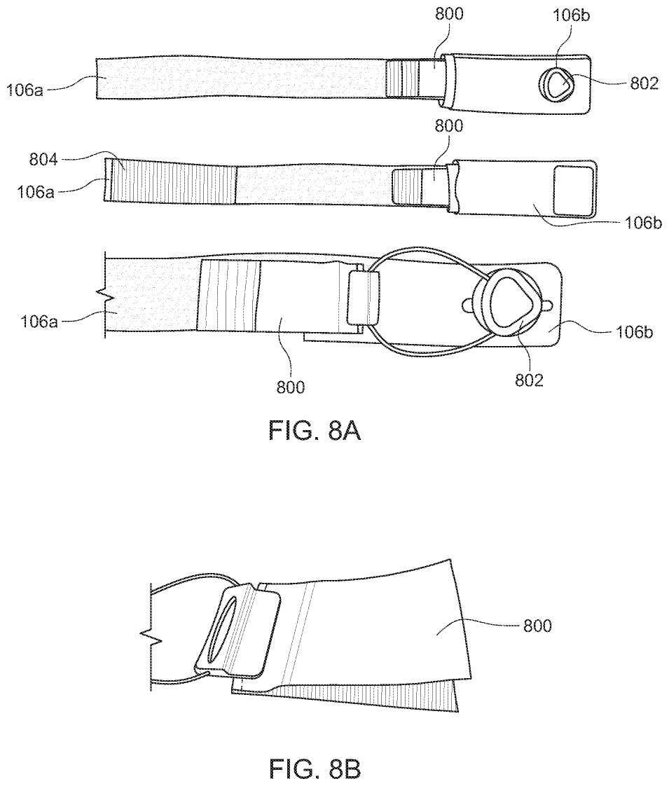

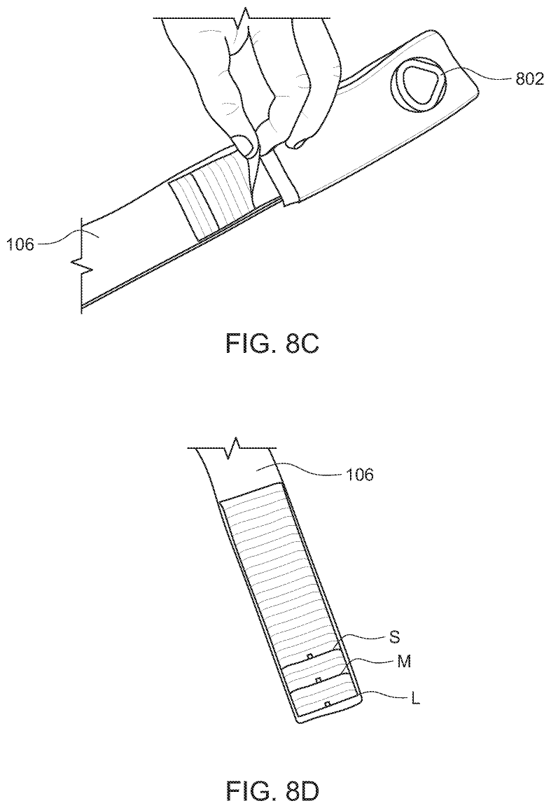

The strap adjuster 104 is shown in detail in FIG. 2a. In the embodiment depicted, the strap adjuster 104 is in the form of a panel having an array of holes or windows 120 in a grid like pattern. Vertically adjacent holes or windows 120 in the grid of the strap adjuster 104 are designed to be cut into larger slit 202 and 204 to accommodate the width of straps 106 and 108 at the height where straps 106 and 108 need to be fixed. Again, noting that as the placement of pads for spinal correction is different for every patient, this customization of the strap adjuster 104 enables straps 106 and 108 to be fixed at a customized height to comply with pad positions where corrective force should be applied to a patient. In the present embodiment, the width of straps 106 and 108 is 4 cm which corresponds to a length of 3 adjacent holes. At the height where strap is to be fixed, two vertical slits 202 and 204 are formed from the 3 adjacent holes in each of two columns of the strap adjuster (see FIG. 2b). The two vertically trimmed slits 202 and 204 enable strap 106 to be threaded through the strap adjuster 104 and folded up on itself and fixed in place via a hook and loop fastening system (see item 804 in FIG. 8 showing the hook-and-loop fastening system with strap 106 in the extended and unfastened state) sewn into overlapping faces of straps 106 and 108 (see FIG. 2c). In this way, the strap adjuster 104 thus provides a quick and easy mechanism for the customized placement of straps 106 and 108 for patients with different spinal curvatures and body lengths whilst avoiding time-consuming fabrication processes involved particularly with hard braces. The strap adjuster 104 is formed or otherwise attached to a heat weldable film 208 so that it can easily be applied to the body back portion 102b.

The components for forming the strap adjuster 104 of the present embodiment include a 3 mm thick grid panel and a heat-weldable film. As the strap adjuster is required to withstand a strong pulling force from the straps, it is important to fix the strap adjuster 104 in position without displacement. To do so, the grid panel of the strap adjuster is 3D-printed from thermoplastic filaments onto the heat-weldable film and is subsequently heat-pressed on to the center back of the bodice 102.

For 3D-printing on to a heat-weldable film, the thermoplastic filaments should have a melting temperature similar to the temperature at which the heat-weldable film is heat pressed onto the bodice 102 e.g. typically around 140° C. If the melting temperature of thermoplastic filament is too high, then heat-weldable film may be ruptured during the 3D printing process because it is unable to withstand the high temperature. Conversely, if the melting temperature of the thermoplastic filament is too low, the thermoplastic filament may not adequately fuse to the heat-weldable film. In the present case, the heat-weldable film has an adhesive softening temperature of about 127° C., can therefore be readily fused with thermoplastic filaments having a melting point of from the softening temperature of the heat-weldable film but below the melting point of the heat-weldable film.

The skilled person will appreciate that the strap adjuster 104 may be formed from a variety of different materials using a variety of different fabrication techniques. However, the inventors have found that thermoplastics having a melting point within the temperature range of 130° C. to 180° C. (and more preferably from 140° C. to 170° C.) are particularly advantageous for the reasons discussed above. In particular, polylactic acid (PLA) was chosen as the filament for 3D printing the grid panel in the present embodiment since the melting temperature of PLA, typically ranging from 150° C. to 160° C., is slightly above the temperature required for application of the heat-weldable film (about 140° C.) to the bodice 102. Furthermore, PLA has good flexural and tensile strength properties for combating the strong pulling force from the straps. In this example, the flexural strength is 9190 psi (63.3 MPa) and the tensile strength is 5710 psi (39.3 MPa).

A suitable range of values for the flexural strength of the strap adjuster is from about 8000 psi (55.2 MPa) to about 10000 psi (68.9 MPa). Preferably, the flexural strength is from about 8500 psi (58.6 MPa). More preferably the flexural strength is from about 9000 psi (62.1 MPa). Alternatively, or additionally, it is preferred that the flexural strength is up to about 9800 psi (67.6 MPa). More preferably, up to about 9600 psi (66.2 MPa). Most preferably, up to about 9400 psi (64.8 MPa).

A suitable range of values for the tensile strength of the strap adjuster is from about 4000 psi (27.6 MPa) to about 6500 psi (44.8 MPa). Preferably, the tensile strength is from about 4500 psi (31.0 MPa). More preferably the tensile strength is from about 5000 psi (34.5 MPa). Alternatively, or additionally, it is preferred that the tensile strength is up to about 6300 psi (43.4 MPa). More preferably, up to about 6000 psi (41.4 MPa).

Straps 106 and 108 partially extend, at the customized height, from the strap adjuster 104 at least partially around the wearers torso and over the treatment area or areas of the wearer to the front body of the bodice 102 where they are attached to a mounting structure near the center of the body front 102a. Whilst a variety of different mounting structures may be used, the present embodiment illustrates the use of hook-and-loop fasteners since this fastening mechanism provides flexibility in terms of the attachment position for straps 106 and 108. Straps 106 and 108 are configured to overlie treatment pads 118 and to be tightened over treatment pads 118 (and against the wearer's body) to provide a bias force to the treatment pads 118 which in turn provide a corrective force against the wearer's scoliotic spine.

The bias force and the corrective forces can be altered by tightening or loosening straps 106 and 108 over their respective treatment pads 118. To achieve this, the present embodiment makes use of a reel-type closure system having a turnable dial 802 on the second end of straps 106 and 108 so as to be accessible from the body front of the bodice 102 to tighten and/or loosen the straps 106 and 108. A suitable reel-type closure system is the Boa® closure system (see FIG. 8a which shows the Front side (top), rear side (middle) and inner construction (bottom) of the Boa® closure system). The Boa® closure system makes strap tightening easier with less physical effort compared with the current hard braces. Hard braces are fastened by pulling the straps to the prescribed tightness with bare hands and usually this requires a great physical effort and often requires assistance from others in order to tighten the brace. The use of the Boa® closure system instead can help wearers to tighten the strap on themselves by simply turning the dial using one hand and with much less effort.

The second end of the strap has Velcro loop tapes sewn on both sides, so it can be attached in between the v-tape hook fastener 800 of the Boa® closure system (see FIG. 8b). Demarcation lines are also printed on the Velcro loop tapes for marking the length of strap used for different body sizes (see small S, medium M, large L markings in FIG. 8c-8d), i.e. small, medium, large, etc. Hence, customization can be done easily with one strap by trimming it to fit the body size of wearer. The other end of the strap has self-adhered Velcro tape sewn on its rear side for fixing the strap on to the strap adjuster 104.

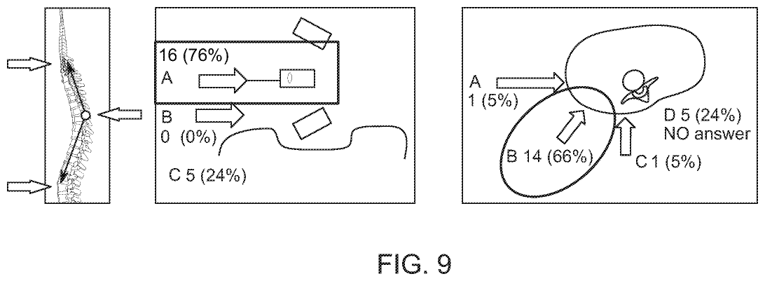

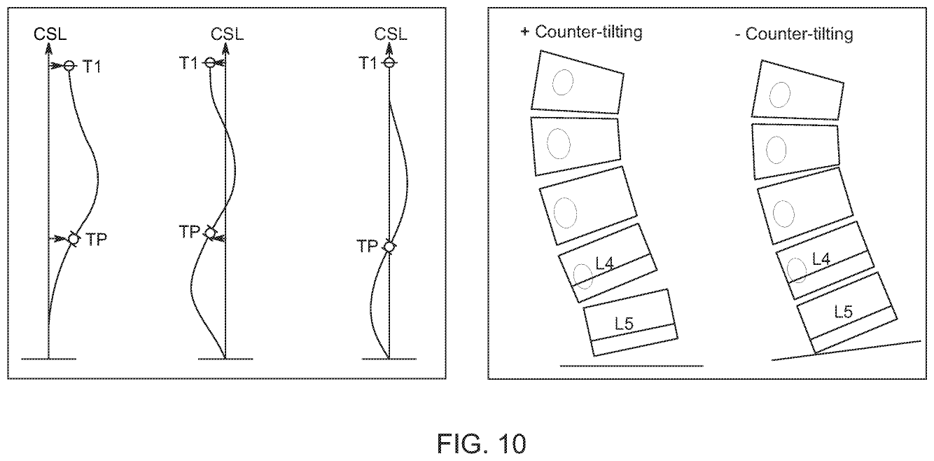

As discussed above, the treatment pads 118 are used to exert corrective forces onto the scoliotic spine. The placement of pads follows biomechanical principles known to those skilled in the art and as discussed in the SOSORT consensus paper on brace action (M. Rigo, S. Negrini, H. R. Weiss, T. B. Grivas, T. Maruyama, and T. Kotwicki, “SOSORT consensus paper on brace action: TLSO biomechanics of correction (investigating the rationale for force vector selection),” Scoliosis, vol. 1, pp. 11, 2006) and the Rigo classification (M. Villagrasa, D. Rigo Manuel, and D. Gallo, “A specific scoliosis classification correlating with brace treatment: description and reliability,” Scoliosis, vol. 5, no. 1, pp. 1, 2010). The disclosures of each of these documents is hereby incorporated by reference in their entirety. As suggested by these publications, the three-point system principle, i.e. use of three pressure points to correct scoliotic spine, is adopted. The three pressure points, where the pads should be located, include one at the apex of the main curve and two at the contralateral side of the curve (see FIG. 9 left). The orientation of pad is at the level of apical vertebra (see FIG. 9 middle) with an oblique force vector from dorso-lateral to ventro-medial (see FIG. 9 right). Pads at the pelvic section are either symmetrical, asymmetrical or placed at one side only, which is dependent on the type of spinal curvature, the position of the T1 vertebra and transitional point (TP) on the central sacral line (CSL) (see FIG. 10 left) and also the counter-tilting relationship between the L4 vertebra and the L5 vertebra in the frontal plane (see FIG. 10 right).

A pair of pad holders is designed for holding the treatment pads 118 onto the bodice (see FIGS. 5-6). The pad holders 116 are formed with a layer of heat-welding film 500 and a layer of fabric 502 which is slightly smaller with 3 edges of the heat-welding film showing. To affix/adhere the pad holders onto the bodice, heat-pressing is carried out at a temperature of 140° C. at 6 bar (90 psi) of pressure for 20 seconds. With the use of heat-welding film in the design of pad holder, the location of pad holders 18 is customizable on the bodice 102 to meet the treatment requirements of an individual patient. The design of pad holder also allows treatment pads 118 to be readily attached, interchanged, or removed to accommodate the changing spine conditions of a patient during the treatment period.

The bodice 102 also includes a plurality of latitudinal or vertically oriented elongate pockets 110 for retaining rigid support struts 112 distributed laterally around the bodice 102. These struts aid the wearer to maintain an upright posture and to prevent the bodice 102 from scrunching up when worn so that the components of the bodice 102 are maintained in position. In particular, the illustrated embodiment includes 14 elongate pockets 110 knitted on the body back of the bodice 102 for receiving the rigid support struts 112. These elongate pockets 110 are evenly distributed laterally around the bodice, e.g. 7 rigid support struts 112 on each lateral side.

The bodice 102 is also connectable with a pelvic belt 114 by way of the strap adjuster 104 to stabilize the pelvis and enhance the spinal correction. In the present embodiment the pelvic belt 114 is formed from a 3 mm layer of Neoprene (a foamed rubber also known as polychloroprene that is both durable and exhibits exceptional tensile performance). The neoprene is laminated with a nylon jersey on the face side and UBL fabric on the back side. UBL fabric is a two-way stretch knit loop fabric which is compatible with hook-and-loop fasteners (e.g. Velcro). Treatment pads 118 for the pelvic area include corresponding hook-and-loop fasteners for customized attachment onto the back side of the pelvic belt 114 according to the spinal curvature and body type of the patient. Hook-and-loop fasteners are also used for fastening the pelvic belt at the front 401. As shown in FIG. 4a, elastomeric laces 400 (such as HICKIES®, see FIG. 4b) are inserted through eyelets 402 of the pelvic belt 114 and fastened to holes 206 on a lower portion of the strap adjuster 104 (see FIG. 1b). In this way, strap adjuster 104 acts as a mount for pelvic belt 114 to bodice 102 at a customized height according to the requirements of the patient. Further, since patients with AIS are undergoing puberty and are likely to grow in height during the treatment period, when necessary, the height of pelvic belt 114 can be adjusted by simply fastening to another row of holes in strap adjuster 104. Further, to prevent the pelvic belt from riding up with body movement, leg straps 404 are sewn at the bottom of the pelvic belt for tightening around the patient's thigh (see FIG. 4c-4d).

It will be understood that the invention disclosed and defined in this specification extends to all alternative combinations of two or more of the individual features mentioned or evident from the text or drawings. All of these different combinations constitute various alternative aspects of the invention.

1. A customizable wearable top to treat scoliosis in a person comprising:
a body portion formed from a soft material and being wearable around the upper torso and having a body front and a body back;
a strap adjuster on an outward facing surface of the body back, the strap adjuster configured to, or customizable to, mount a first end of one or more straps at a selectable longitudinal position thereon, such that when worn the straps are extendable around at least a portion of a wearer's upper torso to apply a bias force to a treatment site on the person corresponding to the selectable longitudinal position.

2. The customizable wearable top of claim 1, wherein the strap adjuster has an elongate body that is vertically aligned with the wearable top.

3. The customizable wearable top of claim 2, wherein the elongate body is a panel and the panel comprises or is customizable to comprise a plurality of attachment points to mount the one or more straps at the selectable longitudinal position.

4. The customizable wearable top of claim 1, wherein the elongate body is located in a central portion of the body back and extends from a body back upper portion to a body back lower portion.

5. The customizable wearable top of claim 1, wherein the strap adjuster is configured to, or is customizable to, mount the one or more straps threadably therethrough.

6. The customizable wearable top of claim 1, wherein the strap adjuster comprises a plurality of selectable components which either alone or in combination form one or more mount portions or are customizable in combination to form one or more mount portions for attachment of the one or more straps.

7. The customizable wearable top of claim 6, wherein each selectable component is a hole or a window, and the plurality of selectable components is an array of holes comprising at least two vertically aligned columns of holes.

8. The customizable wearable top of claim 7, wherein the strap adjuster is cuttable to form a first slit from two or more adjacent holes in a first column of the at least two vertically aligned columns, and a second slit from two or more adjacent holes in a second column, and wherein the strap is mountable to the strap adjuster by threading the strap through the first slit and the second slit.

9. The customizable wearable top of claim 1, wherein the strap adjuster is formed from a thermoplastic material having a melting point in the range of 130° C. to 180° C.

10. The customizable wearable top of claim 1, wherein the strap adjuster is heat welded to the body back.

11. The customizable wearable top of claim 1, wherein the wearable top further comprises a front attachment structure on an outward facing surface of the body front for attachment of a second end of the one or more straps, such that each strap is extendable from the strap adjuster to the front attachment structure.

12. The customizable wearable top of claim 1, wherein the wearable top further comprises the one or more straps.

13. The customizable wearable top of claim 12, wherein each of the one or more straps comprise a reel-type closure system having a turnable dial configured to tighten and/or loosen the straps.

14. The customizable wearable top of claim 1, wherein the body back has a receiving surface for the customized placement of one or more treatment pads and/or mounting structures for retaining the one or more treatment pads at a customized location corresponding to the treatment site.

15. The customizable wearable top of claim 1, wherein the receiving surface is for the customized placement of mounting structures for retaining the one or more treatment pads, each mounting structure being a pocket for receiving a treatment, and wherein the receiving surface is formed from a material to which the one or more pockets are heat weldable.

16. The customizable wearable top of claim 1, wherein the wearable top further comprises a plurality of vertically oriented elongate pockets distributed circumferentially around the body portion, each pocket configured to receive a rigid support strut.

17. The customizable wearable top of claim 1, wherein the strap adjuster further comprises mounting structure for receiving a pelvic belt.

18. A method of preparing a customizable wearable top to treat scoliosis in a person, the method comprising:
providing a customizable wearable top comprising:
a body portion formed from a soft material and being wearable around the upper torso and having a body front and a body back; and
a strap adjuster on an outward facing surface of the body back, the strap adjuster structure configured to, or is customizable to, mount a first end of one or more straps at a selectable longitudinal position thereon such that the straps are extendable around at least a portion of a wearer's upper torso to apply a bias force to a treatment site on the person corresponding to the selectable longitudinal position;
selecting a selectable longitudinal position based on a scoliosis diagnosis of the person; and
mounting the first end of the strap to the strap adjuster at the selected longitudinal location.

19. The method of claim 18, wherein the strap adjuster is customizable to mount the first end of one or more straps at the selectable longitudinal position, and the method further comprises:
customizing the strap adjuster to include one or more mounts to mount the first end of the one or more straps at the selectable longitudinal position.

20. The method of claim 19, wherein the strap adjuster is in the form of a vertically oriented panel having an array of holes or windows arranged in at least two columns, and the step of customizing the strap adjuster further comprises:
cutting a portion of the strap adjuster between two or more adjacent holes in a first column of the at least two vertically aligned columns to form a first slit,
cutting a portion of the strap adjuster between two or more adjacent holes in a second column to form a second slit, and
threading the strap through the first slit and the second slit to mount the strap to the strap adjuster.

21. The method of claim 18, further comprising:
attaching one or more treatment pads and/or mounting structures for retaining the one or more treatment pads at a customized location on a body back portion of the wearable top corresponding to the treatment site.

22. The method of claim 21, wherein the step of attaching the one or more treatment pads and/or mounting structures comprises heat welding the one or more treatment pads and/or mounting structures to the body back portion.
